(19) United States
(12) Patent Application Publication
(10) Pub. No.: US 2026/0192083 A1
(43) Pub. Date: Jul. 9, 2026

(54) SYSTEMS AND METHODS FOR MANUFACTURING AN ULTRASOUND CATHETER TIP

(71) Applicant: Biosense Webster (Israel) Ltd., Yokneam (IL)

(72) Inventors: Wyatt Griffin, Irvine, CA (US); Sreekanth Reddy Rampa, Irvine, CA (US); Audrey Vu, Irvine, CA (US); Michael Govea, Irvine, CA (US); Jasson Rodriguez, Irvine, CA (US)

(21) Appl. No.: 19/429,757

(22) Filed: Dec. 22, 2025

Related U.S. Application Data
(60) Provisional application No. 63/742,540, filed on Jan. 7, 2025.

(51) Int. Cl. A61M 25/00

(52) U.S. Cl. CPC A61M 25/001

(57) ABSTRACT

Methods may include disposing a first ultraviolet (UV) adhesive in a first portion of a cavity of a mold. Methods may include disposing an electro acoustic module (EAM) in the first UV adhesive in the cavity. Methods may include disposing a second UV adhesive in a second portion of the cavity of the mold to encapsulate at least a portion of the EAM disposed therein. Methods may include causing UV light to irradiate at least a portion of the mold. The UV light may cause at least the EAM, the first UV adhesive, and the second UV adhesive to form at least a portion of a catheter tip.

### CROSS-REFERENCE TO RELATED APPLICATION

This application is a Nonprovisional of and claims the benefit of provisional Patent Application Serial No. 63/742,540, filed January 7, 2025, which is hereby incorporated by reference in its entirety for any and all purposes.

### BACKGROUND

Currently, design and manufacturing processes for catheter tips involve manual manufacturing steps. The manual manufacturing steps may involve excessive touch time, which may negatively affect time and/or cost associated with the manufacturing process.

Improvements are needed.

### SUMMARY

The following summary is for illustrative purposes only and is not intended to limit or constrain the detailed description.

Methods may include disposing a first ultraviolet (UV) adhesive in a first portion of a cavity of a mold. Methods may include disposing an electro acoustic module (EAM) in the first UV adhesive in the cavity. Methods may include disposing a second UV adhesive in a second portion of the cavity of the mold to encapsulate at least a portion of the EAM disposed therein. Methods may include causing UV light to irradiate at least a portion of the mold. The UV light may cause at least the EAM, the first UV adhesive, and the second UV adhesive to form at least a portion of a catheter tip. Methods may include removing the catheter tip from the mold. Computer systems, apparatus, and/or computer programs recorded on one or more computer storage devices may be configured to perform the actions of the methods.

Systems may include an electro acoustic module (EAM). Systems may include a first ultraviolet (UV) adhesive. Systems may include a second UV adhesive. Systems may include a mold having: a polytetrafluoroethylene (PTFE) shell; and a cavity in the PTFE shell, where the cavity is configured to receive the first UV adhesive, where the cavity is configured to receive the EAM in the first UV adhesive, and where the cavity is configured to receive the second UV adhesive to encapsulate at least a portion of the EAM. Other materials may be used such as clear acrylic. Systems may include a UV light source configured to cause at least the EAM, the first UV adhesive, and the second UV adhesive to form at least a portion of a catheter tip. Computer systems, apparatus, and/or computer programs recorded on one or more computer storage devices may be configured to perform the actions of the methods.

Catheter tips may be created by a process including disposing a first ultraviolet (UV) adhesive in a first portion of a cavity of a mold; disposing an electro acoustic module (EAM) in the first UV adhesive in the cavity; disposing a second UV adhesive in a second portion of the cavity of the mold to encapsulate at least a portion of the EAM disposed therein; causing UV light to irradiate at least a portion of the mold, where the UV light causes at least the EAM, the first UV adhesive, and the second UV adhesive to form at least a portion of the catheter tip; and removing the catheter tip from the mold. Computer systems, apparatus, and/or computer programs recorded on one or more computer storage devices may be configured to perform the actions of the methods.

These and other features and advantages are described in greater detail below.

### BRIEF DESCRIPTION OF THE DRAWINGS

The present disclosure is pointed out with particularity in the appended claims. However, other features of the present disclosure will become more apparent and the present disclosure will be best understood by referring to the following detailed description in conjunction with the accompany drawings in which:

FIG. 1 shows an example environment in which a catheter manufactured using the systems and methods described herein may operate;

FIG. 2 shows a first portion of an example mold according to the systems and methods described herein.

FIG. 3 shows a device, which is the result of partially filling a mold, such as a mold comprising the first portion of the example mold in FIG. 2, with an ultraviolet (UV) adhesive and curing the UV adhesive.

FIG. 4 shows an example catheter tip 400 made using a mold 450 described herein.

FIG. 5 shows a flow diagram of an example method described herein.

The accompanying drawings show examples of the disclosure. It is to be understood that the examples shown in the drawings and/or discussed herein are non-exclusive and that there are other examples of how the disclosure may be practiced.

### DETAILED DESCRIPTION

The present disclosure generally relates to systems and methods for manufacturing an ultrasound catheter tip.

An example manufacturing method may include folding an electro acoustic module (EAM) at a distal end and at a proximal end and securing the folded EAM together through tacking or some other means. The example manufacturing method may include prefilling a mold with ultraviolet (UV) adhesive. The folded EAM may be placed into the mold and fixed into a position. The mold may be filled completely with UV adhesive after the folded EAM is in the position. The UV adhesive may be cured via a UV wand. The EAM and UV assembly may be removed from the mold. Additional curing may be performed.

Current design and manufacturing processes for catheter tips involve manual manufacturing steps, using excessive touch time, which may negatively affect time and/or a cost of goods sold associated with the manufacturing process. Overmolding presents certain challenges such as high temperatures damaging electronic components and moldability issues, such as thin walls and short shot. The systems and methods described herein disclose a more efficient and reliable way to manufacture a reliable (e.g., without damaging electronics) ultrasound catheter tip.

Conventional methods for manufacturing catheter tips that incorporate ultrasound arrays and other electronics often rely on thermoplastic overmolding processes or extensive manual assembly. In an overmolding process, the catheter shaft and the array are placed into a metallic mold, and a molten thermoplastic material is injected at high temperature and high pressure to form the tip. Such processing conditions can subject piezoelectric transducer elements, flexible circuit substrates, and associated position sensors or wiring to temperatures on the order of 150–250 °C and significant mechanical forces. These conditions can cause thermal degradation of adhesives and encapsulants, cracking or depoling of piezoelectric elements, deformation of thin substrates, or breakage of fine conductors, and can result in low yields and inconsistent performance.

Even when thermal damage is avoided, it is difficult to consistently achieve thin, uniform wall thicknesses in small-diameter catheter tips using injection molding. The small cross-sections and long flow paths associated with such tips make it challenging to fully fill the mold cavity without short-shot defects, while also avoiding flashing or excessive packing pressure. For these reasons, many manufacturers resort to manual build-up techniques in which operators individually position components and apply curable adhesives to construct the tip. Such manual methods are time-consuming, require substantial operator skill, and can suffer from variability in placement, adhesive volume, and curing conditions.

The approaches disclosed herein address the foregoing limitations by employing a low-temperature, low-pressure, UV-curable process in combination with a specially configured folded EAM and a mold geometry that includes secondary fill and cure openings. Because the UV-curable polymeric material can be dispensed at or near room temperature and cured using UV light, the EAM and any embedded position sensor may remain in a benign thermal and mechanical environment throughout the manufacturing process. The mold cavity may define the final outer shape of the tip, allowing precise and repeatable geometries, while the secondary openings may facilitate complete filling and curing around the folded EAM. As a result, the disclosed techniques may provide improved yields, reduced rework, thinner and more acoustically transparent tip walls, and support for more complex internal EAM configurations than would be practical with conventional overmolding or manual methods.

In general, the present disclosure is directed to systems, devices, and methods for forming a distal tip for a catheter that includes an embedded electronic array module (EAM), such as an ultrasound array, using a low-temperature, UV-curable molding process. Instead of relying on high-temperature, high-pressure overmolding or labor-intensive manual build-ups, systems and methods described herein may utilize a mold having a cavity corresponding to the desired catheter tip geometry, one or more UV-curable polymeric materials, and directed UV irradiation, to form a polymeric tip body that encapsulates the EAM and a position sensor, in a controlled and repeatable manner.

The present disclosure relates to a configuration of the EAM within the catheter tip. The EAM may have a distal end and a proximal end and is configured to exhibit a first fold adjacent the distal end and a second fold adjacent the proximal end. Opposing portions of the EAM at each fold may be brought into apposition and secured together, for example by tacking with a UV-curable adhesive or by a mechanical fastener, prior to insertion into the mold. This folded configuration may allow a relatively long effective acoustic array to be compacted within the limited length and diameter of a catheter tip and may provide circumferential or multi-directional coverage without increasing the outer diameter of the catheter.

The present disclosure relates to the design of a mold used to form the catheter tip. The mold may comprise a body of polytetrafluoroethylene (PTFE), clear acrylic, or other material that is substantially non-adhesive with respect to the cured polymer and sufficiently transparent at one or more UV wavelengths. The mold may define a main, catheter-tip-shaped cavity and one or more secondary cavities or openings that are in fluid communication with the main cavity. These secondary cavities, which may include vertical bores and lateral indentations, function as secondary fill openings, vent paths, and/or cure holes to facilitate introduction of UV-curable material around the folded EAM, displacement of air from the cavity, and delivery of UV radiation into regions that would otherwise be shaded by the EAM or other internal components. Clear acrylic may provide superior transparency across the 365–405 nanometer (nm) UV range, facilitating line-of-sight curing from multiple directions, while PTFE may provide comparable opacity with improved chemical resistance. The material choice may depend on the selected UV wavelength and curing geometry

The present disclosure concerns a low-temperature UV-curing process that forms the tip body around the EAM. A UV-curable polymeric material may be introduced into the mold, the folded EAM may be positioned in the main cavity, and additional UV-curable material may be added to at least partially or fully encapsulate the EAM. The mold may be irradiated at one or more UV wavelengths, such as at approximately 365 nm, 385 nm, and/or 405 nm, to cure the material and form the tip body. Because curing may be induced by UV light rather than by elevated mold temperature, the process may be conducted at or near ambient temperature and at low pressure, thereby reducing the risk of thermal or mechanical damage to the EAM and any position sensor, while still achieving accurate tip geometry and consistent encapsulation.

The EAM may comprise a flexible circuit substrate carrying an array of ultrasound transducer elements. For example, the EAM may be formed on a polyimide, polyester, or other polymeric flex circuit having a thickness on the order of 25–125 micrometers. A plurality of piezoelectric elements may be mounted along a length of the substrate, which may be between about 10 millimeters (mm) and about 60 mm, with a width between about 1 mm and about 5 mm. The elements may be arranged in a linear array, a curved array, a ring or partial ring, or a multi-row pattern, with an element pitch and number selected according to the desired imaging resolution and field of view. Conductive traces on or within the substrate may provide electrical connections between the elements and proximal electronics.

The EAM may be configured to exhibit a first fold at a distal region and a second fold at a proximal region prior to encapsulation. The first fold may be formed at a location within about 0.5–5 mm of the distal end of the active array by bending the distal portion of the EAM back toward a more proximal portion about a bend radius selected to avoid damage to the substrate and transducer elements. The second fold may be formed at a location within about 0.5–10 mm of the proximal end of the active array by bending a proximal segment toward a central segment. The folds may be formed around a mandrel or fixture having a radius on the order of 1–5 mm, producing a compact, multi-layered EAM configuration that fits within the desired tip length.

To retain the folded configuration, opposing portions of the EAM at each fold may be secured together. A small volume of UV-curable adhesive, for example on the order of 1–10 microliters, may be applied at one or more contact points between overlapping segments of the EAM and briefly exposed to UV light to partially cure the adhesive and “tack” the segments together. The opposing portions may be held together by a mechanical device such as a micro-clip, band, staple, or suture. These securing mechanisms may prevent the EAM from unfolding or shifting during subsequent handling, insertion into the mold, and encapsulation.

The folded EAM configuration may provide several benefits. It allows an effective array length greater than the linear length of the catheter tip to be accommodated in the tip, thereby increasing the number of transducer elements and the effective acoustic aperture without increasing the tip length or diameter. By adjusting the fold positions and orientations, the EAM segments may be arranged to provide different angular directions of emission and reception, enabling circumferential or multi-directional imaging around the catheter. Additionally, because the folded EAM may be pre-assembled and tacked as a unit, it may be handled and placed into the mold as a single component, reducing assembly steps and operator touch time compared to designs that require individual element placement within the tip.

The mold may include a main cavity defined by complementary indentations in first and second mold portions. The main cavity may extend along a longitudinal axis and have a shape corresponding to the desired exterior geometry of the catheter tip. For example, the main cavity may include a generally cylindrical section having a diameter in the range of about 1.5–5.0 mm and a length in the range of about 5–25 mm, optionally merging into a tapered or rounded distal end region. The proximal end of the main cavity may include a stepped or keyed region configured to interface with a catheter shaft or intermediate support structure.

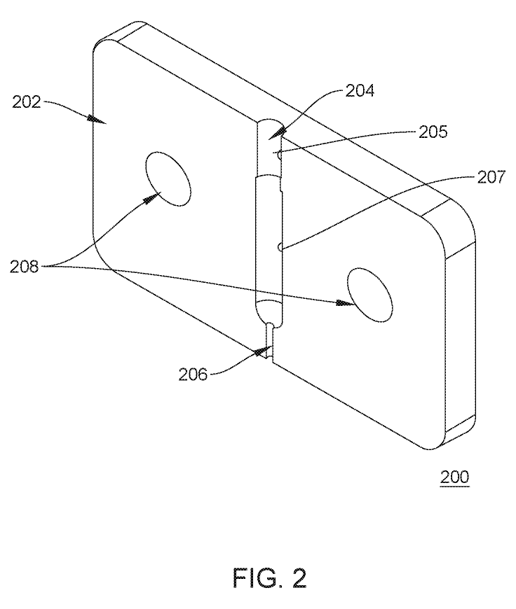

The mold may define one or more secondary cavities or openings that are in fluid communication with the main cavity. These secondary cavities may be provided as vertical bores extending from an outer surface of the mold body toward the main cavity, such as vertical cavities discussed in reference to FIG. 2, and/or as lateral indentations, such as one or more indentations discussed in reference to FIG. 2. These secondary cavities may be positioned to intersect the main cavity at one or more locations along its length, including regions adjacent the folded EAM. The secondary cavities may function as fill and vent paths that permit UV-curable material to enter and exit the main cavity at multiple points, thereby improving flow around the folded EAM and allowing air to escape as the cavity fills. The secondary cavities may function as cure holes, providing line-of-sight access for UV radiation to penetrate into regions of the UV-curable material that may be shadowed by the EAM or other internal structures when viewed only from the proximal or distal ends.

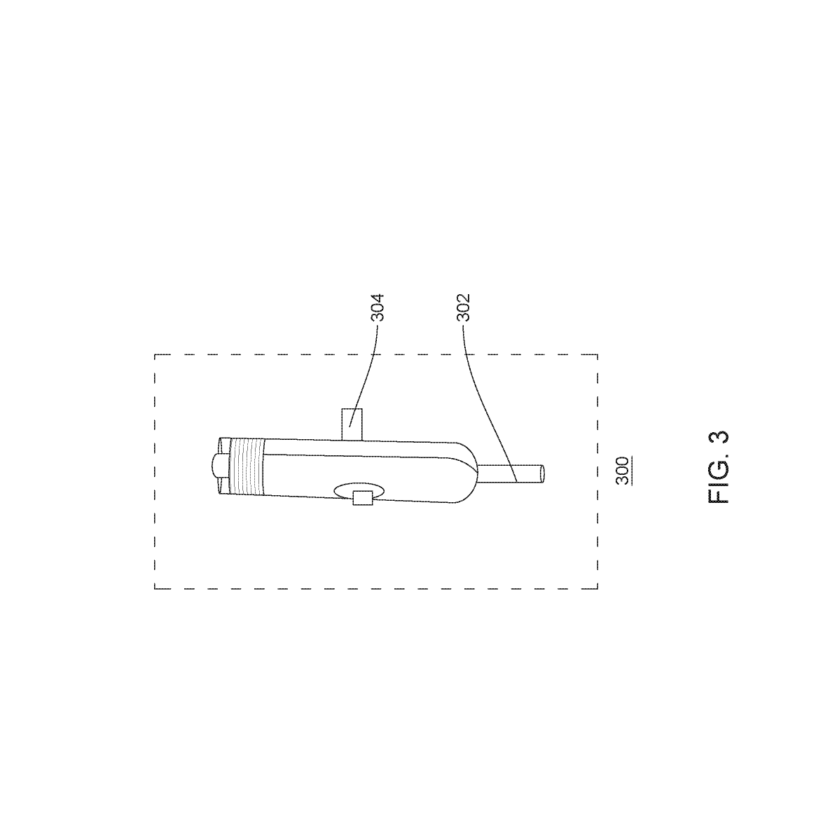

When UV-curable material is introduced into the main cavity and secondary cavities and subsequently cured, the material may solidify into a unified tip body that extends into the secondary cavities, forming protrusions or branches corresponding to the geometries of the secondary cavities. As illustrated in FIG. 3 below, these branches may be remnants or gates associated with the fill and cure paths and are generally not required for the final catheter tip. After the cured tip is removed from the mold, the branches may be removed by cutting, grinding, sanding, polishing, or other finishing operations. The mold design may be be configured such that the branches form at locations that are easily accessible and non-critical to the functional surfaces of the tip, facilitating removal without risk of damage to the encapsulated EAM or the intended external geometry.

The UV-curable polymeric material used to form the catheter tip body may be selected from a variety of acrylate- or methacrylate-based formulations, including urethane acrylates, epoxy acrylates, and combinations thereof, with one or more photoinitiators responsive to radiation in the near-UV region. The material may have a viscosity at 25 °C in the range of about 1,000–20,000 centipoise, a Shore A hardness after cure in the range of about 60–80, and an elongation at break in the range of about 200–600 percent. The material may be substantially transparent or translucent after curing, facilitating visual inspection of the embedded EAM. The material in its uncured state may be lightly colored to assist visual confirmation of filling and transitions to a clearer appearance upon curing, providing an indicator of cure completion.

A first quantity of UV-curable adhesive may be dispensed into the main cavity of the mold to form an initial pool or layer. The folded and secured EAM may be placed into the cavity and partially submerged in the adhesive. Additional adhesive may be introduced, either of the same formulation or of a different formulation, into the main cavity and one or more secondary cavities until the EAM is substantially encapsulated and the cavities are filled to the desired extent. The total dispensed volume may be selected based on the cavity dimensions and the desired wall thickness of the tip.

After filling, the mold may be exposed to UV radiation at one or more wavelengths suitable for the selected adhesive. For example, a light-emitting diode (LED) source operating at a nominal peak wavelength of about 365 nm, 385 nm, or 405 nm and providing an irradiance on the order of 20–80 milliwatts per square centimeter (mW/cm²) at the mold surface may be used. The mold may be irradiated for a period in the range of about 20–180 seconds, which may be delivered in a single exposure or multiple exposures, with light directed through transparent regions of the mold body and through the openings of the secondary and vertical cavities. An initial short exposure (e.g., about 5–20 seconds) may be used to tack-cure the adhesive and immobilize the EAM, followed by a longer exposure to complete the cure. A post-cure step may subsequently be performed after demolding to ensure full conversion and to develop final mechanical properties.

The methods described herein may be implemented using manual, semi-automated, or fully automated equipment. An automated positioning device such as a robotic pick-and-place arm may be used to transfer folded EAMs from a supply tray into the cavities of a plurality of molds held in a fixture. Automated dispensing equipment, including syringe pumps or jet dispensers under electronic control, may be programmed to deliver predetermined volumes of UV-curable material into the main and secondary cavities of each mold with high repeatability. The molds may be transferred, for example by a conveyor or indexing table, into one or more UV curing stations where exposure time and intensity are controlled according to a predefined recipe. Automated demolding and trimming stations may further remove the cured tips, separate them from branches, and perform initial finishing operations. Such automation may reduce manual “touch time,” improve process consistency, and support higher throughput manufacturing compared to purely manual assembly.

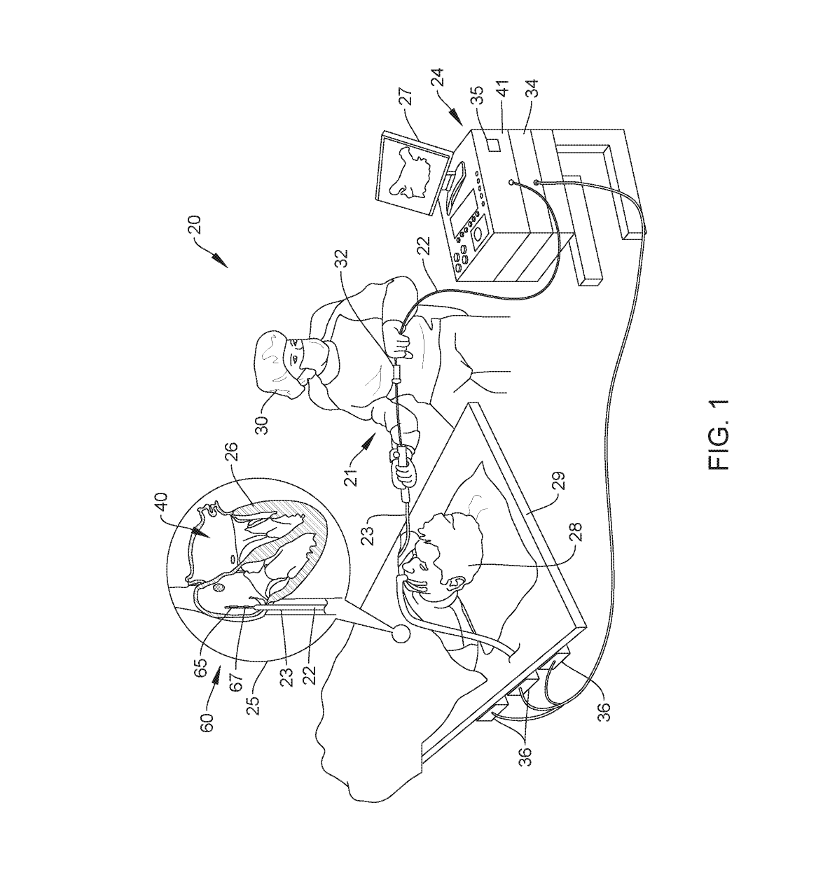

FIG. 1 is a schematic, pictorial illustration of a catheter-based ultrasound imaging system 20 using a catheter 21 with a distal end assembly 60 (shown in an inset 25) comprising an ultrasound array (transducer array) 65 and position sensor 67. The ultrasound array 65 may generate ultrasound images. The position sensor 67 may be preregistered with the ultrasound array 65 of the catheter 21. At least a portion of the distal end assembly 60, such as a portion comprising the ultrasound array 65, may be manufactured using a manufacturing process described herein. The ultrasound array 65 may comprise the EAM. The EAM may comprise the ultrasound array 65.

The position sensor 67 may be configured to output signals indicative of a location and orientation of the ultrasound array 65 inside the organ. A processor (computing device, etc.) 41 of the system 20 may be configured to use the sensor’s signal output to acquire one or more ultrasound images of anatomical structures oriented in various respective orientations relative to ultrasound array 65.

As seen, the distal end assembly 60 may be fitted at the distal end of a shaft 22 of the catheter 21. The shaft 22 may be inserted through a sheath 23 into an organ, such as a heart 26 of a patient 28 lying on a surgical table 29. The proximal end of shaft 22 may be connected to a control console 24.

A physician 30 may navigate the distal end assembly (tip, etc.) 60 of the catheter 21 into the organ of the patient 28 to collect images or prepare for and/or perform a procedure. To rotate the ultrasound array 65 into a required orientation, the physician 30 can, for example, use a manipulator 32 near the proximal end of the catheter 21.

The control console 24 may comprise the processor (computing device, etc.) 41, typically a general-purpose computer, with suitable front end and interface circuits for receiving signals from catheter 21. The console 24 may comprise a driver circuit 34 configured to drive magnetic field generators 36. During the navigation of the distal end 22 in the organ, the console 24 may receive location and orientation signals from the position sensor 67 in response to magnetic fields from external field generators 36. Magnetic field generators 36 may be placed at known positions external to patient 28, e.g., below table 29 upon which the patient 28 is lying. These location and orientation signals may be indicative of the location and orientation of ultrasound-array 65 in a coordinate system of the position tracking system.

The method of location and orientation sensing using external magnetic fields may be implemented in various medical applications, for example, in the CARTO™ system, produced by Biosense Webster, and is described in detail in U.S. Pat. Nos. 6,618,612 and 6,332,089, in PCT Patent Publication WO 96/05768, and in U.S. Patent Application Publications 2002/0065455, 2003/0120150, and 2004/0068178, whose disclosures are all incorporated herein by reference.

An imaged target anatomical structure may be presented to the physician 30 by the processor 41 on a monitor (display, etc.) 27, e.g., as a visual representation of the organ 33.

The processor 41 may be programmed in software to carry out the functions described herein. The software may be downloaded to a memory 35 of the computer in electronic form, over a network, for example, or it may, alternatively or additionally, be provided and/or stored on non-transitory tangible media, such as magnetic, optical, or electronic memory.

The example configuration shown in FIG. 1 is chosen purely for the sake of conceptual clarity. The disclosed techniques may similarly be applied using other contexts where knowledge of heart tissue thickness would be useful.

At least a portion of the distal end assembly 60, such as a portion comprising the ultrasound array 65, may be manufactured using a manufacturing process described herein. The ultrasound array 65 may be configured to exhibit a first fold and a second fold. The ultrasound array 65 may include a distal end and a proximal end. The first fold may be disposed adjacent the distal end and the second fold may be disposed adjacent the proximal end.

The at least the portion of the distal end assembly 60 may be manufactured by disposing a first ultraviolet (UV) adhesive in a first portion of a cavity of a mold. The ultrasound array 65 may be disposed in the first UV adhesive in the cavity. The position sensor 67 may be disposed in the first UV adhesive in the cavity. The at least the portion of the distal end assembly 60 may be manufactured by disposing a second UV adhesive in a second portion of the cavity of the mold to encapsulate at least a portion of the ultrasound array 65 disposed therein. The entire ultrasound array 65 may be encapsulated with the second UV adhesive. At least a portion of the position sensor 67 may be encapsulated with the second UV adhesive. The entire position sensor 67 may be encapsulated with the second UV adhesive. The first UV adhesive may be the same as the second UV adhesive.

The at least the portion of the distal end assembly 60 may be manufactured by causing UV light to irradiate at least a portion of the mold. A UV wand may be used to irradiate the at least the portion of the mold. The UV light may cause the UV adhesive to cure. The UV light may cause at least the ultrasound array 65, the first UV adhesive, and the second UV adhesive to form the at least the portion of the distal end assembly 60. The UV light may cause at least the ultrasound array 65, the position sensor 67, the first UV adhesive, and the second UV adhesive to form the at least the portion of the distal end assembly 60. The at least the portion of the distal end assembly 60 may be removed from the mold.

The distal end assembly 60 may comprise the position sensor 67, such as a magnetic coil sensor or other localization element, positioned adjacent the ultrasound array 65 or at a defined proximal location within the tip body. The position sensor 67 may be electrically connected to the ultrasound array 65 or separate leads, and may be at least partially or fully encapsulated within the UV-cured material together with the ultrasound array 65, allowing simultaneous registration and imaging.

FIG. 2 shows a first portion of an example mold 200. The first portion of the example mold 200 may couple with a second portion (not shown) to form a mold used in the systems and methods described herein. The first portion of the example mold 200 and/or the second portion of the example mold may comprise a material 202. The material 202 may comprise a polytetrafluoroethylene (PTFE) material or acrylic. The material 202 may comprise an acrylic material. The material 202 may comprise a clear acrylic material. A shell may refer to a body of the mold, which may comprise the material 202 and may comprise PTFE, acrylic, and/or other materials.

The first portion of the example mold 200 may comprise a first indention 204. The second portion of the example mold may comprise a second indention. The first indention 204 may correspond to the second indention. When the first portion of the example mold 200 is coupled with the second portion of the example mold, the first indention 204 and the second indention may form a cavity. The cavity may be configured to receive an electro acoustic module (EAM). The EAM may be folded. The cavity may be configured to receive one or more ultraviolet (UV) adhesives. The cavity may be configured to receive a position sensor. The cavity may be configured to receive a UV light to cure UV adhesive disposed in the cavity. The cavity may be configured to form a catheter tip, post, or other components associated with the catheter tip. One or more portions of the cavity may be substantially cylindrical.

The first portion of the example mold 200 may comprise a third indention 206. The second portion of the example mold may comprise a fourth indention. The third indention 206 may correspond to the fourth indention. When the first portion of the example mold 200 is coupled with the second portion of the example mold, the third indention 206 and the fourth indention may form a second cavity configured as a secondary fill and/or a cure hole.

The first portion of the example mold 200 may comprise a first vertical cavity 205 and a second vertical cavity 207. The first vertical cavity 205 and/or the second vertical cavity 207 may act as a secondary fill. The first vertical cavity 205 and/or the second vertical cavity 207 may act as a cure hole. Vertical cavities, such as the first vertical cavity 205 and the second vertical cavity, may be aligned vertically along the first indention 204 of the first portion of the example mold 200. Although shown with two vertical cavities, more vertical cavities, less vertical cavities, or even no vertical cavities are contemplated.

The first portion of the example mold 200 may comprise one or more first apertures 208. The second portion of the example mold may comprise one or more securing components. The one or more first apertures 208 may correspond to the one or more securing components. The one or more securing components may cause the first portion of the example mold 200 and the second portion of the example mold to be coupled together via the one or more first apertures 208.

The second portion of the example mold may comprise one or more second apertures. The one or more first apertures 208 may correspond to the one or more second apertures. One or more fastening components may correspond to the one or more first apertures 208 and/or the one or more second apertures. The one or more fastening components may be used to cause the first portion of the example mold 200 and the second portion of the example mold to be coupled together via the one or more first apertures 208 and one or more second apertures.

Although described in terms of a first portion of the example mold 200 and a second portion of the example mold, a mold according to the systems and methods described herein may be integrated into a single mold.

FIG. 3 shows a device 300, which is the result of partially filling a mold, such as a mold comprising the first portion of the example mold 200 in FIG. 2, with an ultraviolet (UV) adhesive and curing the UV adhesive. One or more portions of the device 300 may be substantially cylindrical. As shown, the device 300 may comprise one or more branches, such as a first branch 302 and a second branch 304. The branches may be remnant and/or gates from the secondary fills and/or cure holes. For example, the first branch 302 may be created by a secondary fill created by the third indention 206 and the fourth indention. As another example, the second branch 304 may be created by the second vertical cavity 207. The device 300 may be substantially similar to the cavity in the mold.

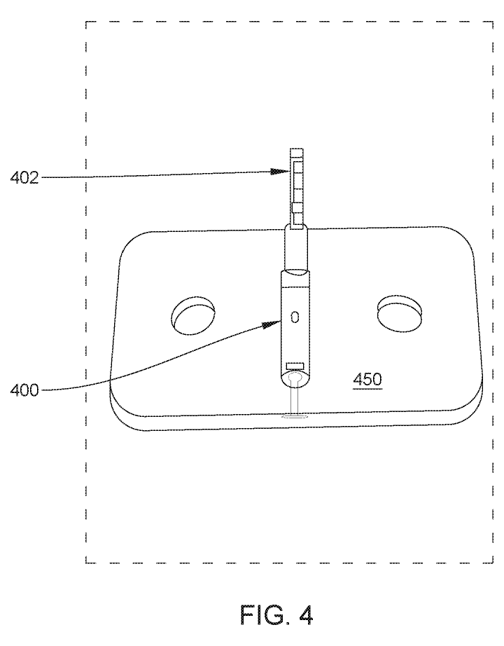

FIG. 4 shows an example catheter tip 400 made using a mold described herein. The catheter tip 400 may comprise an electro acoustic module (EAM) 402. The EAM 402 and ultraviolet (UV) adhesive have been disposed in a cavity of the mold and UV light may have cured the UV adhesive to form the catheter tip 400. The mold may have been separated into a first portion 450 and a second portion. The first portion 450 may comprise the catheter tip 400. The first portion 450 may be to the first portion of the mold 200 described in reference to FIG. 2. The EAM 402 may be folded.

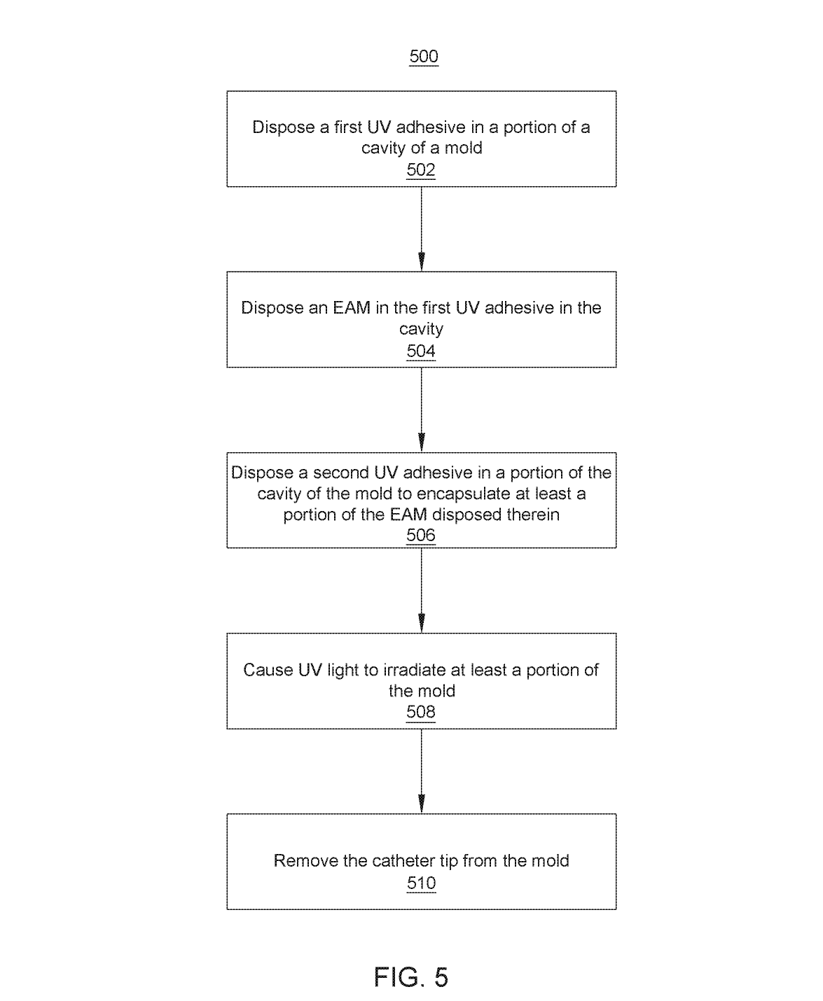

FIG. 5 is a flowchart of an example process. In some implementations, one or more process blocks of FIG. 5 may be performed by a person or a device.

As shown in FIG. 5, the process may include disposing a first ultraviolet (UV) adhesive in a first portion of a cavity of a mold (block 502). The mold may comprise polytetrafluoroethylene (PTFE) material or other material. The mold may comprise acrylic material. The mold may comprise clear acrylic material. The mold may comprise a first portion and a second portion. The first portion may comprise at least one aperture configured to secure the first portion with the second portion. The mold may comprise a secondary fill in communication with the cavity. The cavity may be shaped like a tip of a catheter. The first UV adhesive may be manually disposed in the portion of the cavity of the mold. The first UV adhesive may be automatically disposed in the portion of the cavity of the mold.

As shown in FIG. 5, the process may include disposing an electro acoustic module (EAM) in the first UV adhesive in the cavity (block 504). The EAM may be manually disposed in the first UV adhesive in the cavity. The EAM may be automatically disposed in the first UV adhesive in the cavity.

As shown in FIG. 5, the process may include disposing a second UV adhesive in a second portion of the cavity of the mold to encapsulate at least a portion of the EAM disposed therein (block 506). The first UV adhesive may be the same as the second UV adhesive. The second UV adhesive may be manually disposed in the portion of the cavity of the mold to encapsulate at least the portion of the EAM disposed therein. The second UV adhesive may be automatically disposed in the portion of the cavity of the mold to encapsulate at least the portion of the EAM disposed therein.

At least one of the first UV adhesive and the second UV adhesive may be curable by broad spectrum light. At least one of the first UV adhesive and the second UV adhesive may be curable by light comprising a 365 nanometer wavelength. At least one of the first UV adhesive and the second UV adhesive may be curable by light comprising a 385 nanometer wavelength. At least one of the first UV adhesive and the second UV adhesive may be curable by light comprising a 405 nanometer wavelength. At least one of the first UV adhesive and the second UV adhesive may be curable by the UV light. At least one of the first UV adhesive and the second UV adhesive may be moisture resistant. At least one of the first UV adhesive and the second UV adhesive may comprise a viscosity of 1,100 centipoise (cP) or more. At least one of the first UV adhesive and the second UV adhesive may comprise a viscosity of 1,100 centipoise (cP) or less. At least one of the first UV adhesive and the second UV adhesive, when cured, may comprise a hardness of 70 or harder in accordance with the shore A scale. At least one of the first UV adhesive and the second UV adhesive, when cured, may comprise a hardness of 70 or softer in accordance with the shore A scale. At least one of the first UV adhesive and the second UV adhesive, when cured, may comprise a 600% elongation or more at break. At least one of the first UV adhesive and the second UV adhesive, when cured, may comprise a 600% elongation or less at break.

At least one of the first UV adhesive and the second UV adhesive may comprise a color, and, when cured, may become clear. At least one of the first UV adhesive and the second UV adhesive may comprise a viscosity of 20,000 centipoise (cP) or more. At least one of the first UV adhesive and the second UV adhesive may comprise a viscosity of 20,000 centipoise (cP) or less. At least one of the first UV adhesive and the second UV adhesive may comprise a viscosity of 6,000 centipoise (cP) or more. At least one of the first UV adhesive and the second UV adhesive may comprise a viscosity of 6,000 centipoise (cP) or less. At least one of the first UV adhesive and the second UV adhesive, when cured, may comprise a hardness of 53 or harder in accordance with the shore D scale. At least one of the first UV adhesive and the second UV adhesive, when cured, may comprise a hardness of 53 or softer in accordance with the shore D scale. At least one of the first UV adhesive and the second UV adhesive, when cured, may comprise a hardness of 50 or harder in accordance with the shore D scale. At least one of the first UV adhesive and the second UV adhesive, when cured, may comprise a hardness of 50 or softer in accordance with the shore D scale. At least one of the first UV adhesive and the second UV adhesive, when cured, may comprise a 360% elongation or more at break. At least one of the first UV adhesive and the second UV adhesive, when cured, may comprise a 360% elongation or less at break.

As shown in FIG. 5, the process may include causing UV light to irradiate at least a portion of the mold (block 508). The UV light may cause at least the EAM, the first UV adhesive, and the second UV adhesive to form at least a portion of a catheter tip. The UV light may be from a light-emitting diode (LED) source. The UV light may comprise a 365 nanometer wavelength. The UV light may comprise a 385 nanometer wavelength. The UV light may originate from a UV wand. The UV light may comprise a 405 nanometer wavelength.

As shown in FIG. 5, the process may include removing the catheter tip from the mold (block 510). The catheter tip may be manually removed from the mold. The catheter tip may be automatically removed from the mold.

Optionally, the process may include configuring the EAM to exhibit a first fold and a second fold. The EAM may include a distal end and a proximal end. The first fold may be disposed adjacent the distal end and the second fold may be disposed adjacent the proximal end. A first portion of the EAM before the first fold may be secured to a second portion of the EAM after the first fold. The first portion may be pinned to the second portion. The first portion may be tacked to the second portion. A third portion of the EAM before the second fold may be secured to a fourth portion of the EAM after the second fold. The third portion may be pinned to the fourth portion. The third portion may be tacked to the fourth portion. The first fold may be manually created. The first fold may be automatically created by a device. The second fold may be manually created. The second fold may be automatically created.

Although FIG. 5 shows example blocks of the process, in some implementations, the process may include additional blocks, fewer blocks, different blocks, or differently arranged blocks than those depicted in FIG. 5. Additionally, or alternatively, two or more of the blocks of the process may be performed in parallel.

### EXAMPLE CLAUSES

Example Clause 1: A method may include: disposing a first ultraviolet (UV) adhesive in a first portion of a cavity of a mold; disposing an electro acoustic module (EAM) in the first UV adhesive in the cavity; disposing a second UV adhesive in a second portion of the cavity of the mold to encapsulate at least a portion of the EAM disposed therein; causing UV light to irradiate at least a portion of the mold, where the UV light causes at least the EAM, the first UV adhesive, and the second UV adhesive to form at least a portion of a catheter tip; and removing the catheter tip from the mold.

Example Clause 2: The method of Example Clause 1, where the first UV adhesive is the same as the second UV adhesive.

Example Clause 3: The method of Example Clause 1 or Example Clause 2, where at least one of the first UV adhesive and the second UV adhesive is curable by broad spectrum light.

Example Clause 4: The method of any one of Example Clauses 1-3, where at least one of the first UV adhesive and the second UV adhesive is curable by light may include a 385 nanometer wavelength.

Example Clause 5: The method of any one of Example Clauses 1-4, where at least one of the first UV adhesive and the second UV adhesive is curable by the UV light.

Example Clause 6: The method of any one of Example Clauses 1-5, where at least one of the first UV adhesive and the second UV adhesive is moisture resistant.

Example Clause 7: The method of any one of Example Clauses 1-6, where at least one of the first UV adhesive and the second UV adhesive may include a viscosity of 1,100 centipoise (cP) or more.

Example Clause 8: The method of any one of Example Clauses 1-7, where at least one of the first UV adhesive and the second UV adhesive may include a viscosity of 1,100 centipoise (cP) or less.

Example Clause 9: The method of any one of Example Clauses 1-8, where at least one of the first UV adhesive and the second UV adhesive, when cured, may include a hardness of 70 or harder in accordance with the shore A scale.

Example Clause 10: The method of any one of Example Clauses 1-9, where at least one of the first UV adhesive and the second UV adhesive, when cured, may include a hardness of 70 or softer in accordance with the shore A scale.

Example Clause 11: The method of any one of Example Clauses 1-10, where at least one of the first UV adhesive and the second UV adhesive, when cured, may include a 600% elongation or more at break.

Example Clause 12: The method of any one of Example Clauses 1-11, where at least one of the first UV adhesive and the second UV adhesive, when cured, may include a 600% elongation or less at break.

Example Clause 13: The method of any one of Example Clauses 1-12, where at least one of the first UV adhesive and the second UV adhesive may include a color, and, when cured, becomes clear.

Example Clause 14: The method of any one of Example Clauses 1-13, where at least one of the first UV adhesive and the second UV adhesive may include a viscosity of 20,000 centipoise (cP) or more.

Example Clause 15: The method of any one of Example Clauses 1-14, where at least one of the first UV adhesive and the second UV adhesive may include a viscosity of 20,000 centipoise (cP) or less.

Example Clause 16: The method of any one of Example Clauses 1-15, where at least one of the first UV adhesive and the second UV adhesive may include a viscosity of 6,000 centipoise (cP) or more.

Example Clause 17: The method of any one of Example Clauses 1-16, where at least one of the first UV adhesive and the second UV adhesive may include a viscosity of 6,000 centipoise (cP) or less.

Example Clause 18: The method of any one of Example Clauses 1-17, where at least one of the first UV adhesive and the second UV adhesive, when cured, may include a hardness of 53 or harder in accordance with the shore D scale.

Example Clause 19: The method of any one of Example Clauses 1-18, where at least one of the first UV adhesive and the second UV adhesive, when cured, may include a hardness of 53 or softer in accordance with the shore D scale.

Example Clause 20: The method of any one of Example Clauses 1-19, where at least one of the first UV adhesive and the second UV adhesive, when cured, may include a hardness of 50 or harder in accordance with the shore D scale.

Example Clause 21: The method of any one of Example Clauses 1-20, where at least one of the first UV adhesive and the second UV adhesive, when cured, may include a hardness of 50 or softer in accordance with the shore D scale.

Example Clause 22: The method of any one of Example Clauses 1-21, where at least one of the first UV adhesive and the second UV adhesive, when cured, may include a 360% elongation or more at break.

Example Clause 23: The method of any one of Example Clauses 1-22, where at least one of the first UV adhesive and the second UV adhesive, when cured, may include a 360% elongation or less at break.

Example Clause 24: The method of any one of Example Clauses 1-23, where the mold may include polytetrafluoroethylene (PTFE) material.

Example Clause 25: The method of any one of Example Clauses 1-24, where the mold may include a first portion and a second portion.

Example Clause 26: The method of any one of Example Clauses 1-25, where the first portion may include at least one aperture configured to secure the first portion with the second portion.

Example Clause 27: The method of any one of Example Clauses 1-26, where the mold may include a secondary fill in communication with the cavity.

Example Clause 28: The method of any one of Example Clauses 1-27, further comprising configuring the EAM to exhibit a first fold and a second fold, where the EAM may include a distal end and a proximal end, and where the first fold is disposed adjacent the distal end and the second fold is disposed adjacent the proximal end.

Example Clause 29: A system may include: an electro acoustic module (EAM); a first ultraviolet (UV) adhesive; a second UV adhesive; a mold may include: a polytetrafluoroethylene (PTFE) shell; and a cavity in the PTFE shell, where the cavity is configured to receive the first UV adhesive, where the cavity is configured to receive the EAM in the first UV adhesive, and where the cavity is configured to receive the second UV adhesive to encapsulate at least a portion of the EAM; and a UV light source configured to cause at least the EAM, the first UV adhesive, and the second UV adhesive to form at least a portion of a catheter tip.

Example Clause 30: The system of Example Clause 29, where the first UV adhesive is the same as the second UV adhesive.

Example Clause 31: The system of Example Clause 29 or Example Clause 30, where at least one of the first UV adhesive and the second UV adhesive is curable by broad spectrum light.

Example Clause 32: The system of any one of Example Clauses 29-31, where at least one of the first UV adhesive and the second UV adhesive is curable by light may include a 385 nanometer wavelength.

Example Clause 33: The system of any one of Example Clauses 29-32, where at least one of the first UV adhesive and the second UV adhesive is curable by the UV light.

Example Clause 34: The system of any one of Example Clauses 29-33, where at least one of the first UV adhesive and the second UV adhesive is moisture resistant.

Example Clause 35: The system of any one of Example Clauses 29-34, where at least one of the first UV adhesive and the second UV adhesive may include a viscosity of 1,100 centipoise (cP) or more.

Example Clause 36: The system of any one of Example Clauses 29-35, where at least one of the first UV adhesive and the second UV adhesive may include a viscosity of 1,100 centipoise (cP) or less.

Example Clause 37: The system of any one of Example Clauses 29-36, where at least one of the first UV adhesive and the second UV adhesive, when cured, may include a hardness of 70 or harder in accordance with the shore A scale.

Example Clause 38: The system of any one of Example Clauses 29-37, where at least one of the first UV adhesive and the second UV adhesive, when cured, may include a hardness of 70 or softer in accordance with the shore A scale.

Example Clause 39: The system of any one of Example Clauses 29-38, where at least one of the first UV adhesive and the second UV adhesive, when cured, may include a 600% elongation or more at break.

Example Clause 40: The system of any one of Example Clauses 29-39, where at least one of the first UV adhesive and the second UV adhesive, when cured, may include a 600% elongation or less at break.

Example Clause 41: The system of any one of Example Clauses 29-40, where at least one of the first UV adhesive and the second UV adhesive may include a color, and, when cured, becomes clear.

Example Clause 42: The system of any one of Example Clauses 29-41, where at least one of the first UV adhesive and the second UV adhesive may include a viscosity of 20,000 centipoise (cP) or more.

Example Clause 43: The system of any one of Example Clauses 29-42, where at least one of the first UV adhesive and the second UV adhesive may include a viscosity of 20,000 centipoise (cP) or less.

Example Clause 44: The system of any one of Example Clauses 29-43, where at least one of the first UV adhesive and the second UV adhesive may include a viscosity of 6,000 centipoise (cP) or more.

Example Clause 45: The system of any one of Example Clauses 29-44, where at least one of the first UV adhesive and the second UV adhesive may include a viscosity of 6,000 centipoise (cP) or less.

Example Clause 46: The system of any one of Example Clauses 29-45, where at least one of the first UV adhesive and the second UV adhesive, when cured, may include a hardness of 53 or harder in accordance with the shore D scale.

Example Clause 47: The system of any one of Example Clauses 29-46, where at least one of the first UV adhesive and the second UV adhesive, when cured, may include a hardness of 53 or softer in accordance with the shore D scale.

Example Clause 48: The system of any one of Example Clauses 29-47, where at least one of the first UV adhesive and the second UV adhesive, when cured, may include a hardness of 50 or harder in accordance with the shore D scale.

Example Clause 49: The system of any one of Example Clauses 29-48, where at least one of the first UV adhesive and the second UV adhesive, when cured, may include a hardness of 50 or softer in accordance with the shore D scale.

Example Clause 50: The system of any one of Example Clauses 29-49, where at least one of the first UV adhesive and the second UV adhesive, when cured, may include a 360% elongation or more at break.

Example Clause 51: The system of any one of Example Clauses 29-50, where at least one of the first UV adhesive and the second UV adhesive, when cured, may include a 360% elongation or less at break.

Example Clause 52: The system of any one of Example Clauses 29-51, where the mold may include a first portion and a second portion.

Example Clause 53: The system of any one of Example Clauses 29-52, where the first portion may include at least one aperture configured to secure the first portion with the second portion.

Example Clause 54: The system of any one of Example Clauses 29-53, where the mold may include a secondary fill in communication with the cavity.

Example Clause 55: The system of any one of Example Clauses 29-54, where the EAM is configured to exhibit a first fold and a second fold, where the EAM may include a distal end and a proximal end, and where the first fold is disposed adjacent the distal end and the second fold is disposed adjacent the proximal end.

Example Clause 56: A catheter tip created by a process may include: disposing a first ultraviolet (UV) adhesive in a first portion of a cavity of a mold; disposing an electro acoustic module (EAM) in the first UV adhesive in the cavity; disposing a second UV adhesive in a second portion of the cavity of the mold to encapsulate at least a portion of the EAM disposed therein; causing UV light to irradiate at least a portion of the mold, where the UV light causes at least the EAM, the first UV adhesive, and the second UV adhesive to form at least a portion of the catheter tip; and removing the catheter tip from the mold.

Example Clause 57: The catheter tip of Example Clause 56, where the first UV adhesive is the same as the second UV adhesive.

Example Clause 58: The catheter tip of Example Clause 56 or Example Clause 57, where at least one of the first UV adhesive and the second UV adhesive is curable by broad spectrum light.

Example Clause 59: The catheter tip of any one of Example Clauses 56-58, where at least one of the first UV adhesive and the second UV adhesive is curable by light may include a 385 nanometer wavelength.

Example Clause 60: The catheter tip of any one of Example Clauses 56-59, where at least one of the first UV adhesive and the second UV adhesive is curable by the UV light.

Example Clause 61: The catheter tip of any one of Example Clauses 56-60, where at least one of the first UV adhesive and the second UV adhesive is moisture resistant.

Example Clause 62: The catheter tip of any one of Example Clauses 56-61, where at least one of the first UV adhesive and the second UV adhesive may include a viscosity of 1,100 centipoise (cP) or more.

Example Clause 63: The catheter tip of any one of Example Clauses 56-62, where at least one of the first UV adhesive and the second UV adhesive may include a viscosity of 1,100 centipoise (cP) or less.

Example Clause 64: The catheter tip of any one of Example Clauses 56-63, where at least one of the first UV adhesive and the second UV adhesive, when cured, may include a hardness of 70 or harder in accordance with the shore A scale.

Example Clause 65: The catheter tip of any one of Example Clauses 56-64, where at least one of the first UV adhesive and the second UV adhesive, when cured, may include a hardness of 70 or softer in accordance with the shore A scale.

Example Clause 66: The catheter tip of any one of Example Clauses 56-65, where at least one of the first UV adhesive and the second UV adhesive, when cured, may include a 600% elongation or more at break.

Example Clause 67: The catheter tip of any one of Example Clauses 56-66, where at least one of the first UV adhesive and the second UV adhesive, when cured, may include a 600% elongation or less at break.

Example Clause 68: The catheter tip of any one of Example Clauses 56-67, where at least one of the first UV adhesive and the second UV adhesive may include a color, and, when cured, becomes clear.

Example Clause 69: The catheter tip of any one of Example Clauses 56-68, where at least one of the first UV adhesive and the second UV adhesive may include a viscosity of 20,000 centipoise (cP) or more.

Example Clause 70: The catheter tip of any one of Example Clauses 56-69, where at least one of the first UV adhesive and the second UV adhesive may include a viscosity of 20,000 centipoise (cP) or less.

Example Clause 71: The catheter tip of any one of Example Clauses 56-70, where at least one of the first UV adhesive and the second UV adhesive may include a viscosity of 6,000 centipoise (cP) or more.

Example Clause 72: The catheter tip of any one of Example Clauses 56-71, where at least one of the first UV adhesive and the second UV adhesive may include a viscosity of 6,000 centipoise (cP) or less.

Example Clause 73: The catheter tip of any one of Example Clauses 56-72, where at least one of the first UV adhesive and the second UV adhesive, when cured, may include a hardness of 53 or harder in accordance with the shore D scale.

Example Clause 74: The catheter tip of any one of Example Clauses 56-73, where at least one of the first UV adhesive and the second UV adhesive, when cured, may include a hardness of 53 or softer in accordance with the shore D scale.

Example Clause 75: The catheter tip of any one of Example Clauses 56-74, where at least one of the first UV adhesive and the second UV adhesive, when cured, may include a hardness of 50 or harder in accordance with the shore D scale.

Example Clause 76: The catheter tip of any one of Example Clauses 56-75, where at least one of the first UV adhesive and the second UV adhesive, when cured, may include a hardness of 50 or softer in accordance with the shore D scale.

Example Clause 77: The catheter tip of any one of Example Clauses 56-76, where at least one of the first UV adhesive and the second UV adhesive, when cured, may include a 360% elongation or more at break.

Example Clause 78: The catheter tip of any one of Example Clauses 56-77, where at least one of the first UV adhesive and the second UV adhesive, when cured, may include a 360% elongation or less at break.

Example Clause 79: The catheter tip of any one of Example Clauses 56-78, where the mold may include polytetrafluoroethylene (PTFE) material.

Example Clause 80: The catheter tip of any one of Example Clauses 56-79, where the mold may include a first portion and a second portion.

Example Clause 81: The catheter tip of any one of Example Clauses 56-80, where the first portion may include at least one aperture configured to secure the first portion with the second portion.

Example Clause 82: The catheter tip of any one of Example Clauses 56-81, where the mold may include a secondary fill in communication with the cavity.

Example Clause 83: The catheter tip of any one of Example Clauses 56-82, where the mold may include a single integrated body.

Example Clause 84: The catheter tip of any one of Example Clauses 56-83, wherein the process comprises configuring the EAM to exhibit a first fold and a second fold, where the EAM may include a distal end and a proximal end, and where the first fold is disposed adjacent the distal end and the second fold is disposed adjacent the proximal end.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code - it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

Use of the term “about” is intended to describe values either above or below the stated value in a range of approx. +/- 10%; in other embodiments the values may range in value either above or below the stated value in a range of approx. +/- 5%; in other embodiments the values may range in value either above or below the stated value in a range of approx. +/- 2%; in other embodiments the values may range in value either above or below the stated value in a range of approx. +/- 1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A method comprising:
disposing a first ultraviolet (UV) adhesive in a first portion of a cavity of a mold;
disposing an electro acoustic module (EAM) in the first UV adhesive in the cavity;
disposing a second UV adhesive in a second portion of the cavity of the mold to encapsulate at least a portion of the EAM disposed therein;
causing UV light to irradiate at least a portion of the mold, wherein the UV light causes at least the EAM, the first UV adhesive, and the second UV adhesive to form at least a portion of a catheter tip; and
removing the catheter tip from the mold.

2. The method of claim 1, wherein the first UV adhesive is the same as the second UV adhesive.

3. The method of claim 1, wherein the mold comprises:
a first portion and a second portion that cooperate to define the cavity; and
at least one secondary cavity in fluid communication with the cavity, the at least one secondary cavity configured as at least one of a secondary fill path, a vent path, or a cure hole.

4. The method of claim 3, wherein the mold comprises a material selected from the group consisting of polytetrafluoroethylene (PTFE) material, acrylic, and clear acrylic.

5. The method of claim 1, further comprising configuring the EAM to exhibit a first fold and a second fold prior to disposing the EAM in the first UV adhesive in the cavity, wherein the EAM comprises a distal end and a proximal end, and wherein the first fold is disposed adjacent the distal end and the second fold is disposed adjacent the proximal end.

6. The method of claim 5, further comprising securing opposing portions of the EAM together at each of the first fold and the second fold by at least one of tacking with a UV-curable adhesive or mechanically fastening, prior to disposing the EAM in the cavity.

7. The method of claim 1, wherein at least one of the first UV adhesive and the second UV adhesive is curable by UV light comprising a wavelength selected from about 365 nanometers, about 385 nanometers, and about 405 nanometers.

8. The method of claim 1, wherein at least one of the first UV adhesive and the second UV adhesive comprises:
in an uncured state, a viscosity between about 1,100 centipoise and about 20,000 centipoise at 25 °C; and
when cured, a hardness between about 60 and about 80 on the Shore A scale and an elongation at break between about 200% and about 600%.

9. The method of claim 1, further comprising:
automatically dispensing at least one of the first UV adhesive and the second UV adhesive into the cavity using automated dispensing equipment; and
automatically positioning the EAM into the cavity using an automated positioning device.

10. A system comprising:
an electro acoustic module (EAM);
a first ultraviolet (UV) adhesive;
a second UV adhesive;
a mold comprising:
a shell; and
a cavity in the shell, wherein the cavity is configured to receive the first UV adhesive, wherein the cavity is configured to receive the EAM in the first UV adhesive, and wherein the cavity is configured to receive the second UV adhesive to encapsulate at least a portion of the EAM; and
a UV light source configured to cause at least the EAM, the first UV adhesive, and the second UV adhesive to form at least a portion of a catheter tip.

11. The system of claim 10, wherein the shell comprises at least one of polytetrafluoroethylene (PTFE) material or clear acrylic material.

12. The system of claim 10, wherein the mold comprises a first portion and a second portion that cooperate to define the cavity, and wherein the first portion comprises at least one aperture configured to receive a fastening component to secure the first portion with the second portion.

13. The system of claim 10, wherein the mold further comprises at least one secondary cavity in communication with the cavity, the at least one secondary cavity comprising at least one of a vertical cavity aligned along the cavity and a lateral indentation intersecting the cavity, the at least one secondary cavity configured to act as at least one of a secondary fill opening, a vent path, or a cure hole.

14. The system of claim 10, wherein the EAM is configured to exhibit a first fold and a second fold, wherein the EAM comprises a distal end and a proximal end, and wherein the first fold is disposed adjacent the distal end and the second fold is disposed adjacent the proximal end.

15. The system of claim 10, wherein the first UV adhesive is the same as the second UV adhesive.

16. The system of claim 10, wherein at least one of the first UV adhesive and the second UV adhesive is curable by broad spectrum light.

17. A catheter tip created by a process comprising:
disposing a first ultraviolet (UV) adhesive in a first portion of a cavity of a mold;
disposing an electro acoustic module (EAM) in the first UV adhesive in the cavity;
disposing a second UV adhesive in a second portion of the cavity of the mold to encapsulate at least a portion of the EAM disposed therein;
causing UV light to irradiate at least a portion of the mold, wherein the UV light causes at least the EAM, the first UV adhesive, and the second UV adhesive to form at least a portion of the catheter tip; and
removing the catheter tip from the mold.

18. The catheter tip created by the process of claim 17, wherein the EAM to configured to exhibit a first fold and a second fold, wherein the EAM comprises a distal end and a proximal end, and wherein the first fold is disposed adjacent the distal end and the second fold is disposed adjacent the proximal end.

19. The catheter tip created by the process of claim 17, further comprising a position sensor disposed adjacent the EAM and at least partially encapsulated within the portion of the catheter tip.

20. The catheter tip created by the process of claim 17, wherein the catheter tip further comprises at least one cured protrusion extending from a main body of the catheter tip, the at least one cured protrusion corresponding to a branch formed in a secondary cavity of the mold.
